(19) United States
(12) Patent Application Publication
(10) Pub. No.: US 2025/0041575 A1
(43) Pub. Date: Feb. 6, 2025

(54) INFUSED OR IMPREGNATED FABRICS FOR TREATING DERMATOLOGIC CONDITIONS AND/OR FOR PROVIDING THERAPY, AND METHODS RELATED THERETO

(71) Applicant: Heali Medical Corp., Sarasota, FL (US)

(72) Inventors: Heather Sloan, Concord (CA); Enwei Li, Concord (CA)

(21) Appl. No.: 18/794,282

(22) Filed: Aug. 5, 2024

Related U.S. Application Data
(60) Provisional application No. 63/517,757, filed on Aug. 4, 2023.

(51) Int. Cl. A61M 35/00; A61K 8/02; A61K 8/19; A61K 8/67; A61K 8/92; A61Q 19/08

(52) U.S. Cl. CPC A61M 35/10; A61K 8/0208; A61K 8/19; A61K 8/678; A61K 8/922; A61Q 19/08

(57) ABSTRACT

Fabrics infused or impregnated with one or more ingredients for providing therapy and/or for treating or preventing dermatologic conditions. In certain embodiments, fabric which is stretchable in at least one axis, with an adhesive, which is infused or impregnated with such ingredients. In preferred embodiments, anisotropic fabric to which an adhesive is applied—the fabric and adhesive collectively comprising an adhesive tape—which is impregnated or infused with bioactive ingredients. In still other embodiments, methods for providing therapy or treating or preventing dermatologic conditions.

### RELATED APPLICATION DATA

This application is a non-provisional of U.S. Provisional Patent Application No. 63/517,757, filed on Aug. 4, 2023. The entirety of such application is hereby incorporated by reference.

### FIELD OF THE INVENTION

The various apparatus and method inventions described herein relate to fabrics infused or impregnated with one or more ingredients for providing therapy and/or for treating or preventing dermatologic conditions. Some embodiments of the herein disclosed inventions more specifically relate to fabric which is stretchable in at least one axis, with an adhesive, which is infused or impregnated with such ingredients. In more preferred embodiments, the invention relates to anisotropic fabric to which an adhesive is applied—the fabric and adhesive collectively comprising an adhesive tape—which is impregnated or infused with bioactive ingredients. Certain additional embodiments of this invention relate to methods of providing therapy or treating or preventing dermatologic conditions.

### BACKGROUND OF THE INVENTION

As humans age, human skin develop lines, creases, and folds in the skin colloquially known as “wrinkles”. Medically, they are known as rhytides.

It is generally medically accepted that skin wrinkles are the result of skin that has lost its elasticity and/or plumpness, due to reductions in collagen, elastic, and moisture in the skin as humans age. While these biological conditions happen naturally as age progresses, the onset and severity of wrinkles can rapidly advance due to genetic or environmental factors, such as exposure to ultraviolet rays (e.g., exposure to the sun), pollution, diet, stress, and smoking. Although wrinkles can occur anywhere, they are considered to be particularly socially undesirable when present on the face. Consequently, there are entire industries directed towards reducing the appearance of wrinkles, typically for the goal of reducing the appearance of the age of the person being treated. These industries include over-the-counter cosmetics (where consumers can treat themselves at home), pharmaceuticals, and—on the more extreme end of the spectrum—cosmetic surgery.

Stretch marks are effectively the opposite of wrinkles, and typically occur when the skin is stretched too far, or for too long, or both-presenting themselves when the dermis is strained or broken in places. Stretch marks can materialize with skin color changes, in addition to a ‘stretched’ skin appearance, and are typically considered undesirable in any event. Although stretch marks are most commonly associated with pregnant women—developing as a pregnant woman's abdominal skin stretches throughout pregnancy—they can occur (in men or women) as a consequence of any significant weight gain. Like wrinkles, robust markets exist to treat stretch marks, ranging from consumer-level to medical.

Although various treatments exist to attempt to prevent, treat, or even reverse wrinkes and stretch marks, there is a long felt need in the art for economically affordable treatments which are more effective than those currently known in the marketplace. More specifically, although certain creams and oils exist to treat wrinkles and stress marks, such creams and oils provide only one modality or phase of treatment, or they lack bioefficacy, or both. Undesirable for different reasons, surgical techniques are invasive, expensive, and/or risky procedures which some consumers are simply not able or willing to afford or risk.

In view of the above-described drawbacks or problems in the art, it is apparent that there exists a need for new dermatologic therapies and/or treatments which address, overcome, mitigate, and/or solve one or more of the above-described problems and/or drawbacks. It is a purpose of the various invention(s) disclosed herein to fulfill one or more of these needs in the art and/or other needs which will become more apparent to the skilled artisan once given the following disclosure.

### SUMMARY OF THE INVENTION

Generally speaking, this invention addresses the above-described needs in the art by providing apparatus and methods for providing therapy and/or for treating dermatologic conditions, by concurrently supplying mechanical and bioactive modalities of treatment.

In certain embodiments, apparatus and methods are provided for preventing and/or treating stress marks, such as occur during pregnancy or weight gain. In other embodiments, apparatus and methods are provided for preventing and/or treating rhytides or skin wrinkles.

In at least one non-limiting embodiment, there is provided: a therapeutic apparatus providing at least two therapeutic modalities, a mechanical modality and a bioactive modality, comprising: a fabric strip having a length and width dimension and having a first side and a second side; and the fabric strip being elastic in at least one of the length dimension or the width dimension; an adhesive located on the first side of the fabric strip; the adhesive being configured and composed so as to permit the fabric strip to be adhered to human skin; a therapeutic composition located on the first side of the fabric strip, the therapeutic composition comprising: i) magnesium and ii) one or more ingredients selected from the group consisting of hyaluronic acid, collagen, stems cells of green apples, jojoba oil, and tocopherol; and wherein the fabric strip is configured to be applicable to a skin surface to provide a first therapeutic modality, comprising a structural therapeutic modality; and the therapeutic composition is composed to provide a second therapeutic modality, comprising a bioactive modality.

In some of the above-described embodiments directed towards treatment of stretch marks, the therapeutic composition is more specifically comprised of magnesium; jojoba oil; and/or tocopherol. Conversely, in certain alternative embodiments directed towards treatment of wrinkles, the therapeutic composition comprises: magnesium; green apple stem cells, collagen, and/or hyaluronic acid. Although optional, stem cells from Uttwiler Spätlauber apples are preferred when utilized.

In addition to new and useful apparatus, the applicants herein have also conceived of and hereby disclose and claim methods of prevention and/or treatment of dermatologic conditions, such as stretch marks and wrinkles. In at least one of such embodiments directed to treating pregnant women, there is provided: a method of treating a pregnant woman concurrently with two therapeutic modalities, to prevent or reduce appearance of stretch marks, comprising: applying a fabric strip to an abdominal region of a human woman with an adhesive, the fabric strip having a length and width dimension and having a first side and a second side, the adhesive located on the first side of the fabric strip; the adhesive being configured and composed so as to permit the fabric strip to be adhered to human skin; the fabric strip being elastic in the length dimension; the fabric strip being applied by i) adhering a first end of the fabric strip at a first position on a pregnant female human abdomen comprising a first anchor point; ii) stretching a middle portion of the fabric strip and, while the middle portion of the fabric strip is in a stretched condition, adhering a second end of the fabric strip at a second position on the pregnant female human abdomen, spaced apart from the first position and comprising a second anchor point, such that the fabric strip is adhered to the pregnant female human abdomen in a stretched condition; wherein the space between the first and second anchor points is an area of the pregnant female human abdomen having forces from pregnancy being applied in at least a first force direction, and wherein the stretched condition of the fabric strip causes the fabric strip to apply counter-forces to an area of the pregnant female human abdomen in at least a second force direction which is substantially opposite the direction of the first force direction; and wherein a skin contacting portion of the fabric strip, in contact with the pregnant female human abdomen, includes an infused therapeutic composition comprising: i) magnesium and ii) jojoba oil, and tocopherol.

In preferred embodiments of the methods to treat (or prevent) stretch marks, the fabric strips are secured with adhesive in specific locations—in a stretched condition—to provide counter-forces which at least partially counter the weight of an unborn baby carried by the pregnant female human abdomen; lift the fascia (or the skin and muscle from the fascia); and/or counter the stretched condition of the skin of the pregnant female human abdomen.

In an alternative embodiment, there is provided a method, intended to prevent or treat dermatologic wrinkles concurrently with mechanical and bioactive modalities, comprising: applying a fabric strip to a face region of a human with an adhesive, the fabric strip having a length and width dimension and having a first side and a second side, the adhesive located on the first side of the fabric strip; the adhesive being configured and composed so as to permit the fabric strip to be adhered to human skin; the fabric strip being elastic in the length dimension; the fabric strip being applied by i) adhering a first end of the fabric strip at a first position on a human face comprising a first anchor point; ii) stretching a middle portion of the fabric strip and, while the middle portion of the fabric strip is in a stretched condition, adhering a second end of the fabric strip at a second position on the human face, spaced apart from the first position and comprising a second anchor point, such that the fabric strip is adhered to the human face in a stretched condition;

wherein the first and the second anchor points are selected to tension skin of the face to which the fabric strip is adhered in a direction which relieves a wrinkled skin or facial tension condition; and wherein a skin contacting portion of the fabric strip, in contact with the human face, includes an infused therapeutic composition comprising: i) magnesium and ii) green apple stem cells, collagen, and hyaluronic acid. Preferably, the fabric strips comprise silk weave, and more preferably a synthetic silk weave. However, in optional embodiments, the fabric strips are comprised of cotton weaves.

In preferred embodiments, the fabric strips are elastic in at least one direction and may, indeed, be elastic in all directions. However, in the more preferred embodiments, the fabric strips are anisotropic in that they are elastic in one direction only (e.g., in a length direction, but not width), or at least different in elasticity characteristics in different directions.

Magnesium, in the above-described embodiments, is supplied to provide synergy and/or to improve bio-efficacy (or bioactivity) through improved skin penetration and therefore biologic delivery. In preferred embodiments, the amount of magnesium utilized is an amount by weight of approximately 1.25%-25%. Other amounts, including those described elsewhere herein, may also be utilized depending on the intended application and outcome.

When used herein, the term “fabric strip” shall be construed broadly, as the strip may be provided in innumerable configurations depending on the desired therapy intended. Thus, the fabric strips may be in patch form or in tape form, for example. They may also be provided in sheets which may be cut or which may be pre-cut or pre-scored so that cut or scored portions can be removed to obtain desired fabric strip (or path or tape) shapes. Likewise, the strips can packaged to be sold in sheet form or they may be configured into rolls, so that desired strip (or tape) lengths can be unrolled to be cut or torn or simply removed (in the case of pre-cut rolls) depending on the condition to be treated (or prevented).

The shape and size of the impregnated or infused kinesiology tape is not particularly limited. In some embodiments, the kinesiology tape has a rolled configuration, which may be unrolled for application. In other embodiments, the kinesiology tape may be cut in predetermined shapes and sizes.

The kinesiology tape may be provided in a variety of preselected patterns and preselected colors.

The materials of manufacture are not particularly limited. The substrate may be any suitable fabric known in the art, such as cotton, silk, or a synthetic material. Suitable adhesives or glues will not lose their adhesive properties when in contact with magnesium salts including magnesium chloride.

In some embodiments, the adhesive surface of the tape may have a backing paper or release layer.

Magnesium infused into the herein described apparatus is generally blended into the adhesive in an amount of about 1.25 weight percent to about 16 weight percent of the adhesive blend. However, in some cases, magnesium may be present in amounts up to about 50 weight percent of the adhesive blend.

Infused face tape provides all the benefits of natural creams with the added benefit of structural support. The tape of the present subject matter is breathable, latex free, hypoallergenic and all natural. Impregnated face tape is an elastic, breathable kinesiology tape that can hold the skin on the face in place for long periods of time. The impregnation of collagen, magnesium, apple stem cells, and hyaluronic acid helps ameliorate aging effects such as wrinkles, encouraging cell regeneration, collagen production and much more. These ingredients, in the herein described embodiments, are absorbed transdermally through the skin while the elastic tape, simultaneously, supports and lifts skin to reduce facial swelling, muscle activation, and wrinkles.

Hyaluronic acid, when utilized in embodiments described herein, helps with scarring, wound healing, hydration, reducing wrinkles and age lines. Hyaluronic acid may be present in amounts by weight of about 1.25%-25%.

Collagen, when utilized in embodiments described herein, helps with cell production to improve ageing, moisture, skin healing, wrinkles and in the embodiments in which it is used, it is preferably present in amounts by weight of about 1.25%-25%.

Magnesium helps improve skin's overall appearance including helping with acne, collagen production, wrinkle prevention, and more.

Green apple stem cells, when utilized in embodiments described herein, help to reduce the appearance of fine lines and wrinkles and can also help to improve skin texture and tone. This is because green apple stem cells promote the production of new skin cells and increase collagen and elastin production. Green apple stem cells may be present in amounts by weight of about 1.25%-25%.

Impregnated pregnancy tape of the present subject matter is an elastic, breathable, latex free, hypoallergenic, and all-natural kinesiology tape that can be used as added support for the growing pregnant belly to help lift and support the increased weight. Fabric tape infused with a jojoba oil, magnesium and Vitamin E combination allows for transdermal absorption in addition to support.

Vitamin E, when utilized in embodiments described herein, can help ensure skin is moisturized and minimize stretch mark development. Vitamin E may be present in amounts by weight of about 1.25%-5%.

Magnesium can help with electrolyte balance and cramping. Magnesium may be present in amounts by weight of about 1.25%-25%.

Jojoba oil, when utilized in embodiments described herein, can help ensure skin is moisturized and minimize stretch mark development. Jojoba oil may be present in amounts by weight of about 1.25%-50%.

In certain non-limiting embodiments, impregnated pregnancy tape of the present subject matter is an elastic, breathable, latex free, hypoallergenic, and all-natural kinesiology tape that can be used as added support for the growing pregnant belly to help lift and support the increased weight. Kinesiology tape infused with a jojoba oil, magnesium and Vitamin E combination allows for transdermal absorption in addition to support.

Certain specific examples of the invention are now described below with respect to certain non-limiting embodiments thereof as illustrated in the following drawings wherein:

### BRIEF DESCRIPTION OF THE DRAWINGS

FIG. 1 illustrates a perspective view of one example embodiment of the subject invention configured into roll form.

FIG. 2 illustrates an alternative embodiment of the subject invention, in sheet form with pre-cut tape sections.

FIG. 3 illustrates a tape section from FIG. 2 removed from the illustrated pre-cut sheet.

FIG. 4 illustrates one example of facial tape according to the subject invention.

FIG. 5 illustrates one example of pregnancy tape according to the subject invention.

### DETAILED DESCRIPTION OF CERTAIN EXAMPLE EMBODIMENTS

For a more complete understanding of the present invention, reference is now made to the following description of various illustrative and non-limiting embodiments thereof, taken in conjunction with the accompanying drawings in which like reference numbers indicate like features.

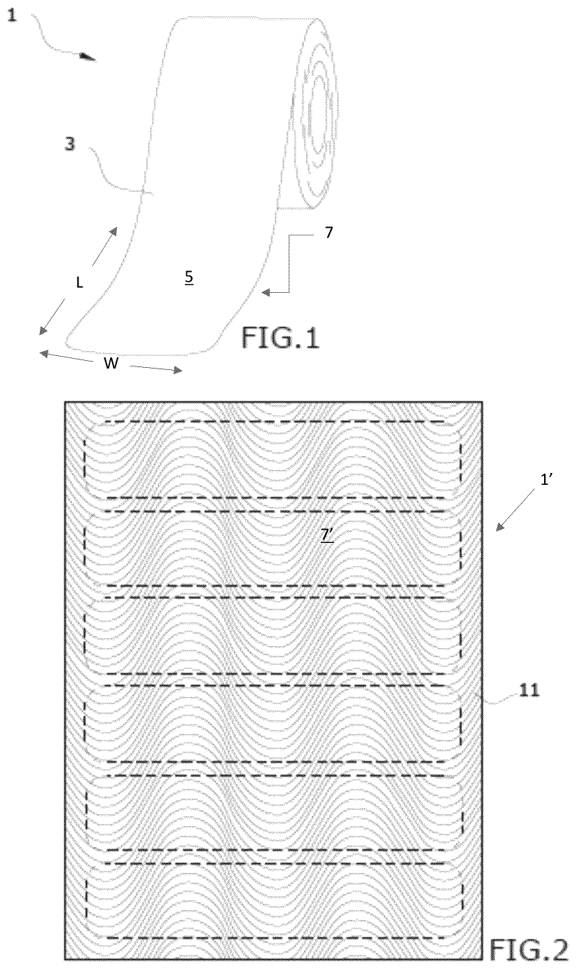

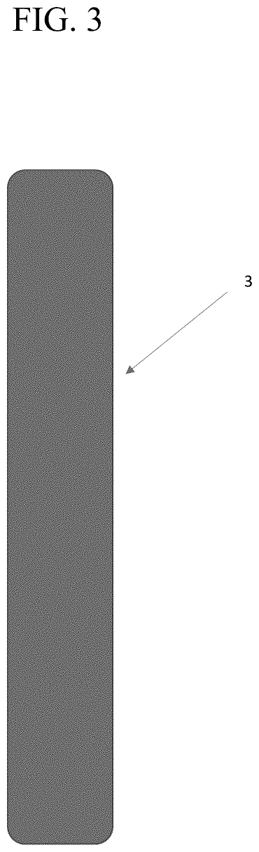

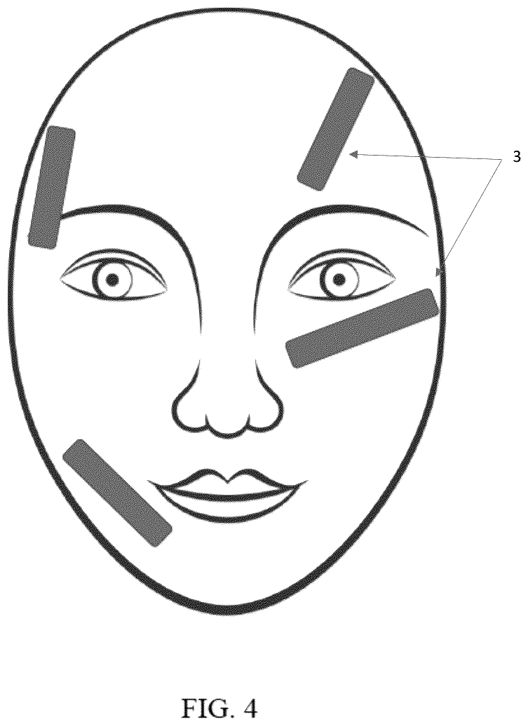

Referring now to FIGS. 1-3, therein is illustrated a non-limiting, example embodiment of a dual modality, therapeutic fabric according to one category of the herein described inventions. In the example embodiment illustrated, the fabric is configured into a tape form, and when combined with infused or impregnated, preferred ingredients, provides mechanical and bioactive treatments and/or therapies simultaneously, thereby providing improvements over the known prior art.

More specifically, tape 1 is comprised of a fabric strip 3 having a length dimension L and width dimension W and having a first side 7 and a second side 5. Tape 1 is preferably elastic in at least either the length dimension L, or in the width dimension W, but in the most preferred embodiments is an anisotropic fabric which is elastic in only one of these dimensions (i.e., the L dimension). Although preferably not elastic in the other dimension, it is recognized—of course—that some elasticity is tolerated and that it is not feasible, and likely impossible, to entirely omit elasticity in a fabric. Accordingly, the description of the fabric as being one-way elastic is intended to be within the accepted definition of anisotropic fabrics and/or to otherwise mean that the fabric is elastic in a first direction and non-elastic in a second direction on a relative basis.

Tape 1, as illustrated, includes an adhesive located on the first side 7 of the fabric strip 3. Although the adhesive layer cannot be seen in FIG. 1, an alternative embodiment of tape, tape 1′ is shown in FIG. 2 (in sheet form) in which second side 7′ and its adhesive 11 is fully visible. In preferred embodiments, the adhesive is a quasi-solid (i.e., firm, gel like material, but applied in thin layers) which is sufficiently elastic such that it does not fail mechanically when stretched with the stretchable fabric. It is configured and composed so as to permit the fabric strip 3 to be adhered to human skin while, prior to being affixed to the skin, also releasably holding a backing paper, which is peeled off when the tape is ready to be used. Application to skin, to obtain an adhered state, to provide or achieve particular therapies is performed according to specific techniques described elsewhere herein below. In the most preferred embodiments, the adhesive has sufficient adhesive strength and durability to remain adhered to the location on the human body to which the tape has been volitionally placed, until the user chooses to remove the tape, by removing the tape mechanically (e.g., by hand, by carefully peeling the tape from the skin). In other words, the tape is preferably configured to withstand movements and activities of a human user, without peeling away on its own prior to the desired treatment end. In some embodiments, it is preferred that the tape remain in place for more than 12 hours, in some cases more than 24 hours, and in other examples, more than 48 hours (and up to one week or more).

In preferred embodiments, fabric strip 3 is comprised of a preferably absorbent, synthetic silk, which is herein defined as a Rayon, Spandex blend. In the most preferred embodiments, the synthetic silk is approximately 95-98% Rayon with the remainder Spandex, with the most preferred embodiments comprising a 97% Rayon, 3% Spandex blend. Such synthetic silk embodiments are preferably used with bioactives such as magnesium combined with jojoba oil, tocopherol (Vitamin E), green apple stem cells, collagen, and/or Hyaluronic acid, as discussed in more detail below. Although synthetic silk is preferably used with the above-described embodiments, a cotton-blend fabric can also be used, preferably comprising approximately 97% cotton and 3% Spandex.

In order to deliver effective doses of bioactives to the skin of a user, the bioactives (magnesium combined with jojoba oil, tocopherol, green apple stem cells, collagen, and/or Hyaluronic acid) are preferably infused or otherwise carried within the adhesive (or glue) layer itself. In the most preferred embodiments, they are also homogenously impregnated so as to achieve uniform exposure of skin to the selected bioactive ingredients. The adhesive (or glue) is preferably applied to the first side 7 of the fabric strip 3 in spaced apart lines, with the spaces between the lines being only fabric (i.e., fabric unadorned with adhesive). While the adhesive is illustrated applied in a repeating wave pattern, other patterns of application can be employed.

Although the herein described inventions preferably provide both bioactive and mechanical therapeutic modalities simultaneously, magnesium, in particular, is supplied in the described embodiments for improving skin health, aiding collagen synthesis, and maintaining skin hydration. Magnesium facilitates collagen synthesis (and thus wrinkle reduction) through enzyme activation and protein synthesis, thus assisting in the prevention or treatment of wrinkles. More specifically, magnesium acts as a cofactor for several enzymes involved in collagen synthesis. One key enzyme is prolyl hydroxylase, which is necessary for stabilizing and cross-linking collagen molecules. This process is instrumental in forming the triple-helix structure of mature collagen fibers. Magnesium also influences the synthesis of proteins, including collagen, by aiding in the transcription and translation processes within cells. Adequate magnesium levels ensure that fibroblasts (the cells responsible for collagen production) can function optimally.

Although use of magnesium has many benefits, magnesium in a chloride solution is ionized and hydrophilic, which means it cannot easily penetrate the lipophilic (fat-loving) stratum corneum. Accordingly, various exemplar embodiments of the inventions described herein combined magnesium with penetrants, such as carrier oils, which aid its penetration into the desired layers of the skin. Specifically, use of carrier oils, such as jojoba oil, can enhance the penetration of active ingredients by disrupting the stratum corneum lipid barrier thus increasing skin permeability. By blending magnesium with jojoba oil, the oil is believed to help to transport magnesium ions through the small, permeable areas of the skin (hair follicles and sweat glands) more effectively.

Although jojoba oil is particularly effective for this purpose, other skin penetrating lipophilic compositions may also be utilized. Vitamin E, for example, is also believed to act as a penetration enhancer, improving the absorption of co-administered substances through the skin. Specifically, Vitamin E is believed to—similar to jojoba oil—enhance skin penetration by changing the structure and properties of the skin's outermost layer, making it more permeable to other substances, including magnesium. In addition to aiding skin permeability, Vitamin E provides the additional benefit of reducing oxidative stress in the skin, which can be beneficial in environments where skin is exposed to UV radiation and pollutants. This protective effect of Vitamin E can help maintain the integrity of skin cells.

In addition to delivering magnesium with penetrant aids, such as jojoba oil and/or Vitamin E, ingredients such as Hyaluronic acid may be utilized to reduce or prevent scarring, for wound healing, hydration, and for reducing wrinkles and age lines. Hyaluronic acid, when utilized, is preferably supplied in amounts by weight of about 1.25%-25%. Likewise, although magnesium assists in collagen production, collagen can also be supplied as an infused ingredient to assist with cell production, reduced the appearance of ageing, increase moisture, promote skin healing, and/or reduce wrinkles. Collagen (preferably plant based) may be present in amounts by weight of about 1.25%-25%.

Green apple stem cells likewise help to reduce the appearance of fine lines and wrinkles and can also help to improve skin texture and tone. This is because green apple stem cells promote the production of new skin cells and increase collagen and elastin production. Accordingly, certain embodiments of the herein described inventions include green apple stem cells infused or impregnated in the fabric tape (i.e., preferably but not necessarily in the glue layer) and may be present in amounts by weight of about 1.25%-25%.

Although, as may be ascertained by the above descriptions, the compositions described herein are useful to treat (or prevent) facial wrinkles and pregnancy related stress marks, the apparatus described herein can also be applied to neck, cleavage or other related areas, and in embodiments where magnesium is utilized to reduced pain, for treatment (for example) of temporomandibular joint dysfunction. In each of the embodiments, however, the primary purpose is to provide a elastic fabric strip, supplied with ingredient infused adhesive, which is configured to be applicable to a skin surface to provide a first structural (or mechanical) therapeutic modality while the infused bioeffective composition provide a second therapeutic modality simultaneously or concurrently.

### Methods of Treatment, Use, and Application

Infused kinesiology tape may be used as follows. A person cleans and prepares a predetermined area of skin so that it is, e.g., oil free and lotion free. Next, if the tape is in roll form, a suitable size length of tape is unrolled from the roll. Or, in the case of pre-cut tapes, a pre-cut segment is unrolled and removed (or detached) from the roll. Next, the paper backing is removed to expose the adhesive layer of the tape. Once the backing tape is removed and the adhesive exposed, the fabric tape can be applied using different techniques to achieve different structural modality objectives.

In one type of treatment for example, to address facial wrinkles, the tape can be applied, such as disclosed in FIG. 4, to pull the facial skin into unwrinkled corrective conditions. Likewise, it can be applied to counteract undesired muscular actions, such as the wrinkling of the eye region (by reducing squinting) or the furrowing of the brow. While applied as such, the fabric tape also lifts and supports the skin, while concurrently delivering bioactive ingredients via the contact of the infused, adhesive glue layer to the skin.

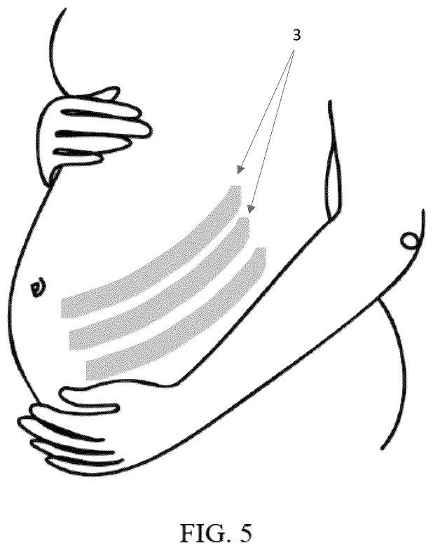

In alternative embodiments directed towards stretch marks and/or relief during pregnancy, such as disclosed in FIG. 5, the fabric tape can be applied using multiple different techniques. For example, the tape can be applied to lift the weight of the unborn baby, while also lifting fascia to increase blood blow and supply of nutrients to the surrounding anatomical areas. In such embodiments, the tape is suitably larger than the facial tapes and is preferably rectangular with rounded corners. In other embodiments, such as for reducing or preventing stretch marks, the fabric tape can be applied by creating two anchor points with a stretch in the middle (between the anchor points). More specifically, the first anchor point can be selected on one side of the stretched skin, and a first end of a tape segment fixed, via pressure and adhesive, on the selected anchor point. The middle of the tape can then be pulled into tension, to stretch the second end of the tape segment to the second anchor point on the opposite side of the stretched skin area. The tape is attached at the second anchor point, like the first anchor point, using pressure to secure the adhesive to the abdominal skin. Applying the tape as such reduces the tension on the abdominal skin (in the area between the two anchor points) while the bioactives are simultaneously infused at skin contact points. The fabric tape may be worn, for example, for up to 1 week including during showering. Pregnancy tape may be applied to the abdomen, as well as anywhere else stretch marks occur, such as breasts. Conversely, as discussed above, face tape strips can be applied to the face as well as to neck and cleavage areas as to help with structural aspects while impregnated ingredients are absorbed transdermally into the skin and muscles.

Once given the above disclosure, many other features, modifications, and improvements will become apparent to the skilled artisan. Such features, modifications, and improvements are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally set forth as limiting, is not intended to impart a narrowing scope to the invention in contravention of the ordinary meaning of the claim terms by which the scope of the patent property rights shall otherwise be determined.

What is claimed is:

1. A therapeutic apparatus providing at least two therapeutic modalities, a mechanical modality and a bioactive modality, comprising:
a fabric strip having a length and width dimension and having a first side and a second side; and said fabric strip being elastic in at least one of said length dimension or said width dimension;
an adhesive located on said first side of said fabric strip; said adhesive being configured and composed so as to permit said fabric strip to be adhered to human skin;
a therapeutic composition located on said first side of said fabric strip, said therapeutic composition comprising: i) magnesium and ii) one or more ingredients selected from the group consisting of hyaluronic acid, collagen, stems cells of green apples, jojoba oil, and tocopherol; and
wherein said fabric strip is configured to be applicable to a skin surface to provide a first therapeutic modality, comprising a structural therapeutic modality; and said therapeutic composition is composed to provide a second therapeutic modality, comprising a bioactive modality.

2. The therapeutic apparatus according to claim 1 wherein said therapeutic composition comprises: magnesium; jojoba oil: and tocopherol.

3. The therapeutic apparatus according to claim 2 wherein said fabric strip comprises a silk weave.

4. The therapeutic apparatus according to claim 3 wherein said fabric strip is anisotropic.

5. The therapeutic apparatus according to claim 1 wherein said therapeutic composition comprises: magnesium; green apple stem cells, collagen, and hyaluronic acid.

6. The therapeutic apparatus according to claim 5 wherein said fabric strip comprises a silk weave.

7. The therapeutic apparatus according to claim 6 wherein said fabric strip is anisotropic.

8. The therapeutic apparatus according to claim 2 wherein said magnesium is provided in an amount by weight of approximately 1.25%-25%.

9. The therapeutic apparatus according to claim 5 wherein said magnesium is provided in an amount by weight of approximately 1.25%-25%.

10. A method of treating a pregnant woman concurrently with two therapeutic modalities, to prevent or reduce appearance of stretch marks, comprising:
applying a fabric strip to an abdominal region of a human woman with an adhesive, said fabric strip having a length and width dimension and having a first side and a second side, said adhesive located on said first side of said fabric strip; said adhesive being configured and composed so as to permit said fabric strip to be adhered to human skin; said fabric strip being elastic in said length dimension; said fabric strip being applied by i) adhering a first end of said fabric strip at a first position on a pregnant female human abdomen comprising a first anchor point; ii) stretching a middle portion of said fabric strip and, while said middle portion of said fabric strip is in a stretched condition, adhering a second end of said fabric strip at a second position on the pregnant female human abdomen, spaced apart from said first position and comprising a second anchor point, such that said fabric strip is adhered to the pregnant female human abdomen in a stretched condition; wherein the space between the first and second anchor points is an area of the pregnant female human abdomen having forces from pregnancy being applied in at least a first force direction, and wherein the stretched condition of said fabric strip causes said fabric strip to apply counter-forces to an area of the pregnant female human abdomen in at least a second force direction which is substantially opposite the direction of said first force direction; and
wherein a skin contacting portion of said fabric strip, in contact with the pregnant female human abdomen, includes an infused therapeutic composition comprising: i) magnesium and ii) jojoba oil, and tocopherol.

11. A method of preventing or treating dermatologic wrinkles, concurrently with mechanical and bioactive modalities, comprising:
applying a fabric strip to a face region of a human with an adhesive, said fabric strip having a length and width dimension and having a first side and a second side, said adhesive located on said first side of said fabric strip; said adhesive being configured and composed so as to permit said fabric strip to be adhered to human skin; said fabric strip being elastic in said length dimension; said fabric strip being applied by i) adhering a first end of said fabric strip at a first position on a human face comprising a first anchor point; ii) stretching a middle portion of said fabric strip and, while said middle portion of said fabric strip is in a stretched condition, adhering a second end of said fabric strip at a second position on the human face, spaced apart from said first position and comprising a second anchor point, such that said fabric strip is adhered to the human face in a stretched condition;
wherein the first and said second anchor points are selected to tension skin of the face to which the fabric strip is adhered in a direction which relieves a wrinkled skin or facial tension condition; and
wherein a skin contacting portion of said fabric strip, in contact with the human face, includes an infused therapeutic composition comprising: i) magnesium and ii) green apple stem cells, collagen, and hyaluronic acid.

12. The method according to claim 10 wherein said magnesium is provided in an amount by weight of approximately 1.25%-25%.

13. The method according to claim 11 wherein said magnesium is provided in an amount by weight of approximately 1.25%-25%.

14. The method according to claim 12 wherein fabric comprises an anisotropic silk weave.

15. The method according to claim 13 wherein fabric comprises an anisotropic silk weave.

16. The method according to claim 10 wherein said counter-forces applied are selected from the group consisting of: i) forces to at least partially counter the weight of an unborn baby carried by the pregnant female human abdomen; ii) forces to lift the fascia; and iii) forces to counter the stretched condition of the pregnant female human abdomen.

17. The therapeutic apparatus according to claim 5 wherein said fabric strip is configured into a tape.

18. The therapeutic apparatus according to claim 5 wherein said fabric strip is configured into a patch.

19. The therapeutic apparatus according to claim 18 wherein said fabric strip comprises a cotton weave,

20. The therapeutic apparatus according to claim 5 where said green apple stem cells are stem cells from Uttwiler Spätlauber apples.
